(19) United States
(12) Patent Application Publication
(10) Pub. No.: US 2002/0115055 A1
(43) Pub. Date: Aug. 22, 2002

(54) Gender differentiation of bovine sperm cells

(76) Inventor: Marcos Fernando de Resende Matta, Del Castilho (BR)

Correspondence Address: DEWITT ROSS & STEVENS, S.C., Firstar Financial Centre, Suite 401, 8000 Excelsior Drive, Madison, WI, 53717-1914, US

(21) Appl. No.: 09/965,017

(22) Filed: Sep. 27, 2001

(30) Foreign Application Priority Data
Sep. 28, 2000 (BR) P1-10005045

(51) Int. Cl. A01N 1/02; G01N 33/53; G01N 33/567; A01K 67/027

(52) U.S. Cl. 435/2; 435/7.2; 800/15

(57) ABSTRACT

A method to make an immuno-differentiation of bovine sperm cells using monoclonal antibodies against male specific antigen, 17.18 kDa protein, associated to proteins of the classic pathway complement system is described. Monoclonal antibodies directed towards a gender-specific protein associated to the complement, provoke cellular rupture. For this methodology to be useful, an elimination of the alternative pathway of the complement is necessary. The sperm cells are then put in contact with a monoclonal antibody specifically for the gender-marking protein. Thus, the classic pathway complement system causes selected rupture of the marked sperm cells. The sperm cells of the treated semen are then ready to be used in artificial insemination, cryo-preservation or “in vitro” fertilization.

### BIBLIOGRAPHICAL REFERENCES

Full citations of the references in the following description can be found in the ibliography preceding the claims.

### FIELD OF THE INVENTION

This invention is directed to a method of selecting sperm based on sex. The instant invention describes a method of utilizing monoclonal antibodies against male specific antigens around 17.18 kDa protein, associated with an action of complementing the classic pathway, in order to select for female encoded sperm. This method has practical applications in agricultural in vitro fertilization and artificial insemination, particularly in cattle.

### BACKGROUND OF THE INVENTION

In the dairy and beef industry, farmers and ranchers desire a certain sex of animal. Dairy farmers are primarily in need of female animals, and may only use a few males for stud, if at all. Often farmers forego the cost of male animals entirely, solely depending on artificial insemination techniques. The cattle ranching industry prefers male animals, because they tend to be larger in overall size, and often in quality of meat/hide. With sex selection, a farmer can choose the desired sex, and have a much better than 50-50 chance of the desired sex of offspring.

Sex selection techniques also benefit the bottom line of the farmer. Typically, a farmer has to use the top 40-50% of the herd for breeding to replace the desired sex of the animal. With sex selection, the farmer need only use 20-25% of his herd, because the desired sex is selected for. The farmer also need not worry about getting rid of or destroying the additional animals of the unwanted sex, because few would be produced. This technology is also applicable to the breeding of other animals including sheep, goats, pigs, and the like for agricultural purposes, as well as in horses for racing, polo, and show. Unfortunately, current sex selection techniques for in vitro fertilization in animals do not produce a satisfactory sex ratio, and remain too expensive to be used widely in the industry. Thus, a method for accurately separating sperm before performing costly in vitro procedures is desirable.

### DESCRIPTION OF PRIOR ART

Animal semen contains approximately an equal amount of sperm cells with Y-chromosomes and X-chromosomes. Fertilization of an oocyte with a Y-chromosome produces a male, while sperm cells with the X- chromosome produces a female. Various methods have been applied in the attempt to raise the frequency of embryos for a desired gender. However, none of the currently available methodologies prove to be efficient in determining the exact quantities of semen necessary for artificial insemination. The first methods used to obtain fractions of semen rich in spermatozoa X- or Y-chromosome was a separation by motility (Kaiser et al., 1974), and sedimentation by density (Soupart, 1975). These methods assumed that a sperm cell with a Y- carrying chromosome had less density and higher mobility. However, due to a variable morphology of spermatozoa, these techniques did not obtain success in enriching populations of sperm cells.

Another method of separation was based on the differences in the composition of DNA among the population of spermatozoa. Sperm cells containing X chromosomes should be heavier than the carriers of the Y chromosome, and using this data, researchers have worked the separation of populations by flow cytometry. The main problems with this technique is the high cost, the impossibility of obtaining large fractions of semen differentiated, and the minimal differences in the contents of DNA between spermatozoa X and Y (Spaulding, U.S. Pat. No. 5,660,997). These problems render separation difficult, and expensive. As a result, these techniques are not commercially viable for the agricultural industry.

Immunology methods have also been used in the separation of sperm cells X and Y. These methods are based on the fact that RNA polymerase of spermatozoa is able to transcribe genome haploid (Morre, 1971). Likewise the differences between antigens of the surface contained in a different population of spermatozoa could be used in their separation. Despite this evidence, no efficient method has been presented yet.

One of the most studied antigens in pre-determining gender has been the antigen male-specific denominated H-Y. Indirect evidence suggests that the antigen H-Y is an antigen of the surface present in males and not in females. Based on this antigen, two patents utilizing antibody anti-H-Y to obtain rich fractions of X spermatozoa were granted (Bryant, U.S. patent No. 4,191,749 and Bryant, U.S. patent No. 4,448,767). However no one has been able to confirm the results obtained by these patents. Some researchers suggest that a protein H-Y is not synthesized by the spermatozoa Y, but absorbed on its surface (Garner, 1984). Accordingly, Hoope and Koo (1984) show that both spermatozoa X and Y react with antibody H-Y. Moreover, these authors show that the antibody declines its ability to react with sperm cells when mature, implicating a mask or under expression of the antigen. Another supposition is that the techniques used and their experimental delineation have failed to demonstrate the presence of antigens (Moore and Gledhill, 1988).

Silvers et al. (1982) suggest the presence of antigens detect serological differences from H-Y using experiments of transplants rejection. Based on these experiments, these researchers suggest the designation SDMA (serologically defined male antigen). These results have been confirmed in numerous laboratories, where researchers, using a variety of methods and different types of male specific antidote (Reilly and Goldberg, 1991).

Considering the expense and difficulty associated with these experiments, and the success rates of the above techniques, a preferable technology is described herein.

### SUMMARY OF THE INVENTION

The instant invention describes a method for gender differentiation of bovine spermatozoa wherein monoclonal antibodies against a 17.18 kDa protein associated with the alternative pathway are eliminated by inactivation of the B protein by inactivating the alternative pathway of the complement system. This is done by incubating heated guinea pig serum and ascitic fluid with sperm cells and monoclonal antibodies. These monoclonal antibodies are specific enough to recognize only male specific antigens, and thus, the classic pathway complement system causes selected rupture of the marked male sperm cells. Therefore, a majority of the resulting live sperm are female, and when used in artificial insemination, cryo-preservation or in vitro fertilization, the majority of the resulting offspring will be female. The following experiments illustrate the procedure and efficacy of the instant invention.

### DETAILED DESCRIPTION OF THE INVENTION

The instant invention describes a method of utilizing monoclonal antibodies against 17.18 kDa protein male specific antigens associated with an action of the classic pathway complement. To develop this technology, various types of mammalian serum were tested. Whether the serum presented antibodies with non-specific reactions (cross reactions) could also eliminate the sperm cells in a non-specific form was also tested. Some sera presented an alternative pathway of the complement for determined cells and not for others. An example was the case of the chicken serum that presented an alternative pathway against erythrocytes of horse. Cow serum, goats serum, sheep serum, and pig serum present several undesirable reactions for the complement selection. Guinea pig serum presented a strong action of the alternative pathway of complement, killing 100% of the sperm cells in approximately 15 minutes of contact. However, agglutination was not present in these cells, a demonstration that there are no antibodies in the guinea pig serum against the proteins of the bull sperm cells. The following experiments illustrate the procedure and efficacy of the instant invention.

### Experiment I

To build on existing monoclonal antibody work, it was necessary to verify that previous fluorescence experiments utilizing monoclonal antibodies would fail to produce a good technique for separating sperm cells for sex. An experiment was done utilizing two antibodies. The first was a monoclonal antibody, and the second antibody was specific against mouse immunoglobulin, conjugated with particles of iron for later separation by a magnetic column (IMAC). All dead sperm cells were fixated, while all the live sperm cells were eluted using IMAC, to select for female sperm cells. These live sperm cells were then used for in vitro fertilization and the embryos produced were tested by polymerase chain reaction (PCR) analysis utilizing primers to identify the Y chromosome. The results indicated that 65% of the embryos were female, far less than the desired result.

### Experiment II

The following experiment built on prior monoclonal antibody experiments. In this experiment, 10⁷ sperm cells were used for each procedure. To verify the motility of the sperm cell in the diluent (TRIS buffer+yolk), the sperm cells was placed in contact with 100μl of diluent and incubated for one hour. For verification of motility in the presence of the serum, the sperm cells were incubated with 100 μl of guinea pig serum for an equal amount of time. To verify motility in the presence of monoclonal antibodies and guinea pig serum, the sperm cells were initially incubated for 45 minutes with 100 μl of monoclonal antibodies, and washed afterwards by centrifugation at 2000 g and incubated again for an hour with the guinea pig serum. All incubations were done at 37° C.

The source for monoclonal antibodies was ascitic fluid that was previously heated at 52.2° C. by 30 minutes to eliminate the B protein potentially originated from peritoneal macrophages. To eliminate the alternative pathway of the complement, the guinea pig serum was heated to different temperatures, for 30 minutes and placed in contact with sperm cells for 1 or 2 hours and microscope analysis was carried out. Variations among animals of the same species and among animals of different species occurred in relation to the ideal temperature to inactivate B protein. A range of different temperatures was used to inactivate the B protein of the guinea pig serum and to incubate these sera with sperm cells. The primary objective was to observe the inactivation of the alternative pathway and the presence of monoclonal antibodies and to verify the action this serum by the classic pathway of the complement. Reference is made to Table 1 as follows:

TABLE 1

| | | | Motility + antibody |
|---|---|---|---|
| Temperature | Motility % dilute | Motility % serum | % |
| 48° C. | 70% | 0% | 0% |
| 50° C. | 70% | 0% | 0% |
| 51° C. | 70% | 0% | 0% |
| 52° C. | 70% | 70% | 40% |
| 52° C. | 70% | 70% | 70% |

Table 1 illustrates that a range of different temperatures were used to inactivate the B protein of the guinea pig serum and to incubate these sera with sperm cells. The primary objective was to observe the inactivation of the alternative pathway, and the presence of monoclonal antibodies, to verify the action this serum by the classic pathway of the complement

### Experiment III

Sperm cells were then incubated with the heated guinea pig serum and heated ascitic fluid (all at the same time, and separately), using different concentrations of serum and ascitic fluid. Experiments used a concentration of 10⁷ sperm cells with previously washed cells, with the seminal plasma was separated for later use, (except for experimental procedures marked by asterisks). After being washed, the cells were incubated with ascitic fluid for 45 minutes, and washed again to remove any excess of antibodies. These cells were then incubated with the guinea pig serum for one hour. At this point, 100 μl of semen plasma was added to the serum and the same amount of PBS (phosphate-buffered saline) containing Ca⁺⁺ and Mg⁺⁺ and incubated for one hour. After this, the alive cells are separated by PERCOLL gradient and these cells were used for in vitro fertilization procedure. The gender of the resulting embryos was determined by PCR, using the specific primer for Y chromosome. In the procedure marked by an asterisk, the serum, ascitic fluid and PBS were added simultaneously, and in the procedures marked by two asterisks, there was no separation of the seminal plasma and of the volume containing 10⁷ cells was taken out from the ejaculated.

The results obtained with 20 μl of serum and 20 μl of ascitic fluid were similar to those obtained with 100 μl of serum and 100 μlm of ascitic fluid. To verify the efficiency of the technology, PCR analysis and in vitro fertilization was performed. The results are shown in Table 2 as follows:

| | PCR Results - Sex Semen | | | | | | | | | | | | | | | | |
|---|---|---|---|---|---|---|---|---|---|---|---|---|---|---|---|---|---|
| | 20/20 | | 20/30 | | 30/20 | | 30/30 | | 50/50 | | 100/100 | | *50/50 | | **100/100 | | Female |
| Bull | M | F | M | F | M | F | M | F | M | F | M | F | M | F | M | F | (%) |
| 1 | 5 | 17 | | | 3 | 13 | | | | | | | | | 3 | 7 | 77.1 |
| 2 | 2 | 22 | | | 3 | 27 | | | | | | | | | 0 | 14 | 92.6 |
| 3 | 5 | 8 | 4 | 6 | 2 | 5 | 2 | 7 | | | | | | | 2 | 4 | 66.6 |
| 4 | | | 2 | 21 | | | | | 4 | 20 | | | 2 | 8 | 1 | 3 | 85.2 |
| 5 | | | | | | | | | | | | | | | 1 | 3 | 75.0 |
| 6 | | | | | | | | | 2 | 6 | 4 | 9 | 5 | 8 | 2 | 7 | 75.0 |
| 7 | | | | | | | | | | | 0 | 1 | 3 | 14 | 7 | 19 | 77.2 |
| Tot. | 12 | 47 | 6 | 27 | 8 | 45 | 2 | 7 | 6 | 26 | 4 | 10 | 10 | 30 | 16 | 57 | |

| | | | | | | | | |
|---|---|---|---|---|---|---|---|---|
| Fem. | 79.6 | 81.8 | 84.9 | 77.7 | 81.2 | 71.4 | 75.0 | 78.1 |

Table 2 shows that the volume of ascitic fluid and serum utilized in the experimental procedure did not significantly influence the sexing semen, thus the methodology using cellular lyse complement-mediated is sensitive enough, and the antibody is also specific enough to recognize the male specific antigens. The experiment shows that of 308 embryos analyzed, 244 were female and only 64 male, which corresponds to 79.2% success in differentiation. The volume of ascitic fluid and serum utilized in the experimental procedure did not significantly influence the sexing semen. These results show that the methodology using cellular lyse complement-mediated is sensitive enough, and the antibody is also specific enough.

### Experiment IV

Experiment III suggests that the epitope recognized by the monoclonal antibody could vary according to the maturity stage of the sperm cells, and therefore prevent the monoclonal recognition of some male sperm cells, which may be the reason that gender differentiation did not reach 100%. This is clearly a complement dependent cytotoxicity, which was able to rupture all sperm cells marked by antibodies. Additionally, volume variation did not influence the results.

Alternately, using monoclonal antibodies against different epitopes of male specific proteins should give better results and allow gender differentiation closer to 100%.

Based on this, an experiment was performed using the technique of intracytoplasmatic injection of sperm cells (ICSI). In this experiment, “good” sperm cells, with desirable physiologic characteristics, were selected and placed in contact with an oocyte to be fertilized. Results yielded 24 female embryos and only 2 male embryos. These results correspond to 92.3% of gender differentiation.

### Experiment V

The following experiment shows the effect of the monoclonal antibody in the presence of the complement, and without the complement. Sperm cells, placed in contact with only monoclonal antibodies were observed by microscope. Living and swimming sperm cells, in pairs, were observed as being fixed by antibodies. This connection made the sperm cells present a larger force and dispense more energy, leading to their death after a determined period of time (approximately 2 hours). When these sperm cells were placed in the presence of antibodies and complement, it was observed that the sperm cells formed pairs or clusters. However, all of these sperm cells died by action of the complement.

Sperm cells were then placed only in the presence of monoclonal antibodies, and were then used in in vitro fertilization. This process resulted in 25 male embryos and 50 female embryos, a ratio similar to the observed ratio in column IMAC. These results show a limited success of gender differentiating which was significantly improved by the use of the complement, confirming the overall results.

Experimental variation may be related to variations observed among animals, or even variations among procedures. In order to obtain better and more homogenous results the interval of collecting between ejaculations, genetic variations among animals, and maturity of sperm cells should be normalized. Techniques for this are in progress.

These experiments illustrate the process used to find the best method of sex selection. As a result of these trials, it is shown that the inactivation the B protein which activates the alternative pathway and placing the sperm cells in contact with a monoclonal antibody that marks for gender and thereby lysing the male sperm cells, sex selection for all kind of fertilization procedures can be achieved.

It is understood that the invention is not confined to the particular reagents, reactions, and methodologies described above, but embraces all modified and equivalent forms thereof as come within the scope of the following claims.

What is claimed is:

1. A method for gender differentiation of bovine spermatozoa wherein monoclonal antibodies against male specific antigen, 17.18 kDa protein, associated with the classical pathway of the complement system are used after the inactivation of the alternative complement pathway, comprising:
a. incubating heated guinea pig serum with sperm cells and monoclonal antibodies, said monoclonal antibodies being specific enough to recognize only male specific antigens, and
b. selectively rupturing the male sperm cells, leaving a majority of the live sperm cells as female.

2. The method of claim 1 wherein the serum is selected from the group including cow serum, goat serum, sheep serum, pig serum and guinea pig serum.

3. The method of claim 1 wherein the serum is guinea pig serum.

4. The method of claim 1 wherein the serum and ascitic fluid are heated at 52.2° C.+0.3 for 30 minutes.

5. The method of claim 1 wherein the sperm cells is incubated with monoclonal antibodies for 45 minutes at 37° C.

6. The method of claim 1 further comprising following step b. washing the sperm cells to remove excess of antibodies, and then adding the serum and seminal plasma to the sperm and then incubating for 1 hour at 37° C.

7. A method for gender differentiation of bovine spermatozoa wherein monoclonal antibodies against male specific antigen, 17.18 kDa protein, associated with the classical pathway of the complement system are used after the inactivation of the alternative complement pathway, using the following steps:
a. Inactivating the alternative pathway of the complement system by heating guinea pig serum and ascitic fluid at 52.2° C.+0.3 for 30 minutes or purified monoclonal antibodies; and
b. Incubating the sperm cells with monoclonal antibodies, originated from one or more different clones (hybridomas), for 45 minutes at 37° C.; and
c. Washing the sperm cells to remove excess of antibodies, and then adding the inactivated guinea pig serum and seminal plasma to the sperm cell and then incubating for 1 hour at 37° C.

8. The method of claim 7, wherein the B protein in a guinea pig serum inactivates the alternative pathway of the complement.

9. The method of claim 7 comprising eliminating the B protein at temperatures varying from 50° to 53° C.

10. The method of claim 7 comprising eliminating the alternative pathway of the complement by methods selected from column affinity, genetic modification of animals, and utilization of protein H is foreseen in this patent.

11. The method of claim 7, wherein monoclonal antibodies are incubated with the sperm cells for the time necessary for there to be an antigen-antibody binding.

12. The method of claim 7, wherein the serum is incubated for a time sufficient for the reaction of the classic pathway of the complement system to proceed.

13. The method of claim 7 wherein the antibody and complement are incubated simultaneously with the sperm cells.

14. The method of claim 7 wherein the monoclonal antibodies are reacted with different epitopes for better differentiation.
